(19) United States
(12) Patent Application Publication
(10) Pub. No.: US 2022/0301675 A1
(43) Pub. Date: Sep. 22, 2022

(54) SYSTEM AND METHODS FOR MONITORING AND CUSTOMIZING CONSUMPTION OF HERBS

(71) Applicant: GRAMSS CANNABIS LTD, Tel Aviv (IL)

(72) Inventor: Aviel COHEN, Tel Aviv (IL)

(21) Appl. No.: 17/603,924

(22) PCT Filed: Sep. 3, 2020
(86) PCT No.: PCT/IL2020/050964

§ 371 Date: Oct. 14, 2021

Related U.S. Application Data
(60) Provisional application No. 62/895,020, filed on Sep. 3, 2019.

(51) Int. Cl. G16H 20/10; G16H 40/63; G16H 20/60; G16H 10/60

(52) U.S. Cl. CPC G16H 20/10; G16H 40/63; G16H 20/60; G16H 10/60

(57) ABSTRACT

A system for monitoring and customizing herb consumption, comprising: a server computer; at least one digital herb scale for coupling to a mobile computing device for monitoring herb consumption; the server computer is configured to perform the following: obtaining metadata of a person including physical details and herb consumption information gathered by the digital herb scale; based on the metadata, finding a group of at least two persons that have at least one matching parameter in their physical details and herb consumption information; in said group, finding a sub-group of at least two persons that further have an at least one additional matching parameter in their physical details and herb consumption information; customizing a plan for herb consumption for a person based on herb consumption information of at least one other person in said sub-group.

### FIELD OF THE INVENTION

The present invention relates to system and methods for computerized monitoring and customizing the consumption of herbs.

### BACKGROUND OF THE INVENTION

The modern industry of cannabis has revealed thousands of new cannabis strains, with infinite potential, each strain has its own genetics, component level, flavors & effects. Along with medical breakthroughs that are constantly finding new potential in treating different diseases with the cannabis plant, yet not very much is known about adapting the cannabis strains.

Legalization of marijuana for medical and recreational use yielded a fast growing and prosperous industry. In the US for example, marijuana has been approved for medical use in 33 states, as of November 2018, and is used to treat symptoms associated with diseases such as AIDS, cancer and glaucoma. Furthermore, medical marijuana is used worldwide to treat also a number of different conditions, such as Alzheimer's disease, appetite loss, Crohn's disease, eating disorders such as anorexia, Epilepsy, mental health conditions like schizophrenia and posttraumatic stress disorder (PTSD), multiple sclerosis, muscle spasms, Nausea, pain, wasting syndrome (cachexia) and counting.

Cannabis strains are either pure or hybrid varieties of the plant genus Cannabis. Varieties are developed to intensify specific characteristics of the plant, to differentiate the strain for purposes of marketing, or to make it more effective as a drug. The cannabis plant contains more than 100 different chemicals called cannabinoids. Each one has a different effect on the body. Delta-9-tetrahydrocannabinol (THC) and cannabidiol (CBD) are the main chemicals used in medicine.

### SUMMARY OF THE INVENTION

The present invention discloses a system and methods for monitoring and customizing herb consumption. The system typically comprises: a server computer coupled to a database that is configured to store data obtained from client mobile computing devices and a digital herb scale to be coupled in digital communication to a mobile computing device for monitoring herb consumption.

The server computer is configured to obtain and analyze metadata from various client mobile computing devices and to perform the following actions:

obtaining metadata of a person using the system including his physical details and also his herb consumption information gathered by the digital herb scale;

based the metadata, the server computer proceeds at finding a group of at least two persons that have a matching parameter in common in their physical details and herb consumption information;

in the group, the server computer proceeds at finding a sub-group of at least two persons that further have an additional matching parameter in their physical details and herb consumption information;

finally, the server computer proceeds at customizing a plan for herb consumption for a person based on herb consumption information of other persons in that person's sub-group.

The inventive system and methods of the present invention provide means to customize herb consumption plans based on other persons' parameters and their herb consumption usage information.

### BRIEF DESCRIPTION OF THE DRAWINGS

subject matter regarded as the invention is particularly pointed out in the concluding portion of the specification. The invention, however, both as to organization and method of operation, together with objects, features, and advantages thereof, may best be understood by reference to the following detailed description when read with the accompanying drawings in which:

FIG. 1 illustrates a system according to an embodiment of the invention;

FIG. 2A illustrates an isometric exploded view of a herb grinder according to an embodiment of the invention;

FIG. 2B illustrates a cross section view of a herb grinder according to an embodiment of the invention;

FIG. 3A illustrates an isometric exploded view of a herb grinder according to an embodiment of the invention;

FIG. 3B illustrates a cross section view of a herb grinder according to an embodiment of the invention;

FIG. 4 illustrates a networked computer system arrangement according to an embodiment of the invention;

FIG. 5 illustrates a method according to an embodiment of the invention.

FIG. 6 illustrates a block diagram of an app platform according to an embodiment of the invention; and

FIGS. 7A and 7B illustrate flowcharts according to an embodiment of the invention.

### DETAILED DESCRIPTION OF THE PREFERRED EMBODIMENTS

### GRAMSS—Platform

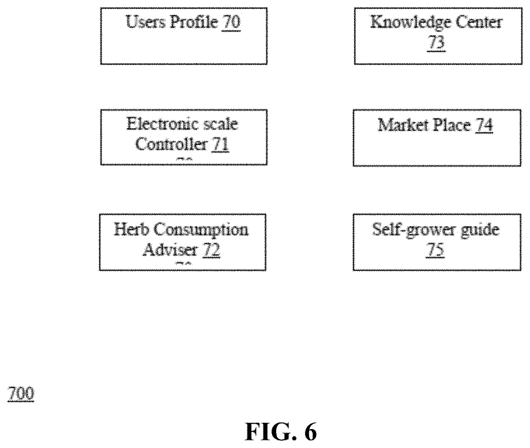

FIG. 6 illustrates a block diagram of an app platform according to an embodiment of the invention.

The app platform 700 may include at least one of the followings: a user's profile 70, an electronic scale controller 71, a herb consumption adviser 72, a knowledge center 73, a market place 74 and a self-grower guide 75.

User's profile 70 may include personal details of a user (such as his name, his residence address, a profile picture etc.), user's physical details, user's herb consumption information and user's contact information.

Electronic scale controller 71 may provide means for controlling an electronic scale of a herb grinder. It may turn on/off the electronic scale, command to perform calibration, display scale results, indicate battery power etc.

The herb consumption adviser 72 may provide tailored recommendations based on user's physical details and/or user's herb consumption information in order to improve and customize user's herb consumption to his specific needs.

The knowledge center 73 may provide a collection of herbs (e.g. Cannabis) related network resources, such as web pages and multimedia content.

The market place 74 may be dedicated to obtaining services, purchasing of items, joining multiple user events and the like. According to one embodiment of the invention user of the app platform may purchase from the market place 76 a specific Cannabis strain that was recommended by the herb consumption adviser 72 based on user's profile 70.

The self-grower guide 75 may provide an extensive tutorial information related to growing herbs. Such information may include growing techniques, explanation on various parameters to be monitored (such as desired humidity levels, optimal temperature, PH levels etc.), plant adjusted nutrients, irrigation frequency, required light exposure—and the like.

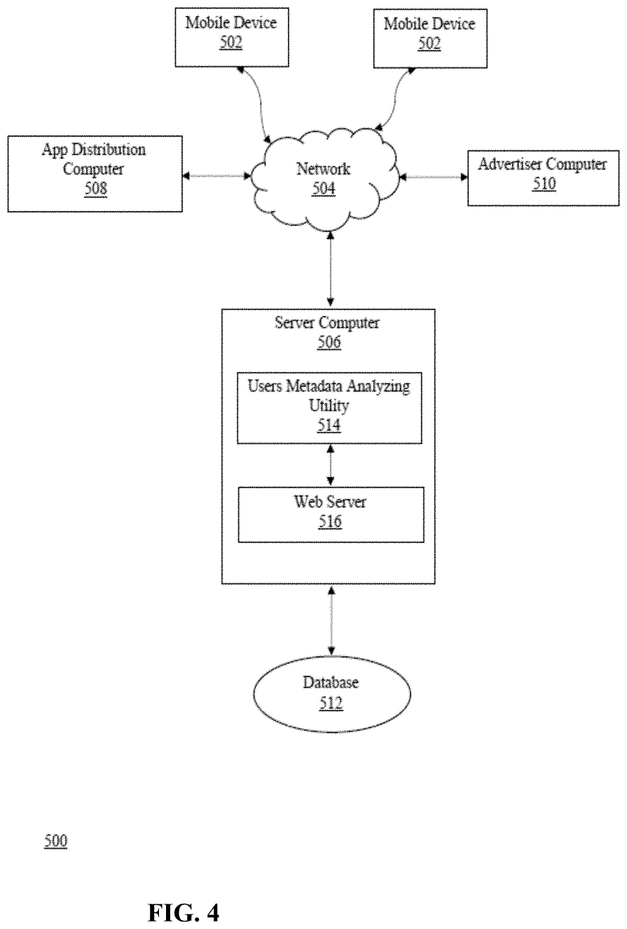

FIG. 4 illustrates a networked computer system arrangement according to an embodiment of the invention.

Optionally, the computer system comprises one or more mobile computing devices 502, a network 504, at least one server computer 506 coupled to a database 512, one or more app distribution computers 508 and one or more advertiser computers 510.

Each of the mobile computing devices 502 comprises a smartphone, tablet computer, netbook computer, laptop computer, or other movable computer. Typically wireless links couple the mobile devices 502 to network 504, which broadly represents one or more local area networks, wide area networks, internetworks, global interconnected internetworks such as the public internet, or a combination thereof. For purposes of illustrating a clear example, two (2) mobile devices 502 are shown in FIG. 4 and one cloud symbol representing network 504 is shown, but practical implementations may use thousands or millions of mobile devices and any number of interconnected networks.

App distribution computer 508 is associated with a developer, author or publisher of a mobile device application computer program or app, or a distribution channel such as a centralized app downloading service; examples include the APPLE APP STORE and GOOGLE PLAY service. The app is a product-related app wherein method of operation and details thereof are illustrated in FIGS. 4-6 and described in detail hereinafter. Typically the app developer, author or publisher desires to provide advertising within the graphical user interface displays of the app, or is willing to enable certain functions, options or features in the app in exchange for display of advertising within the app. Typically the app developer, author or publisher does not have direct contract or compensation relationships with advertisers but relies on server computer 506, and/or an advertising network or advertising service with which it generally does have a contractual relationship with which is supported by the server computer, to provide advertisements on the app. The advertiser computer 510 may serve ads to the app installed on mobile devices 502 directly or the server computer 506 may serve the ads. Users of mobile devices 502 benefit from use of the app and exposure to products or services that may interest them. They also interact with ads served from Network 504.

Server computer 506 may be coupled to a database 512 that is configured to store data obtained from the mobile devices 502.

According to an embodiment of the invention, server computer 506 hosts at least a web server 516 and users metadata analyzing utility 514, which is configured to perform the processes herein associated with obtaining and analyzing metadata obtained from the mobile devices 502.

FIG. 4 illustrates a diagrammatic representation of a system in the exemplary form of a computer system including HW and SW such as e.g. set of instructions, causing the system to perform any one or more of the above techniques. In alternative embodiments, the machine may be connected (e.g. networked) to other machines in a Local Area Network (LAN), an intranet, an extranet, or the Internet. The system may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g. computers) that individually or jointly execute a set (or multiple sets) of instructions to perform any one or more of the methodologies discussed herein.

The exemplary computer system 500 may include a processor. The processor represents one or more general-purpose processing devices such as microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instructions sets, or processors implementing a combination of instruction sets. Processor may also be one or more special-purpose processing devices such as an application specific integrated circuit (ASIC), a field programmable gate array (FPGA), a digital signal processor (DSP), network processor, or the like.

### GRMSS—Consuming

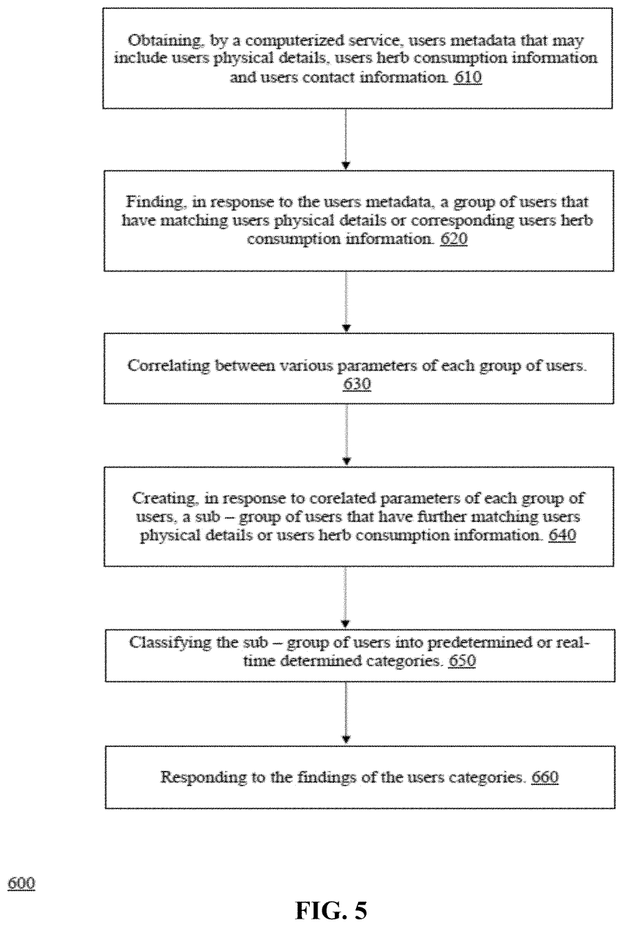

FIG. 5 illustrates a method 600 according to an embodiment of the invention.

Method 600 may start by stage 610 of obtaining, by a computerized service, users metadata that may include users' physical details, users herb consumption information and users contact information. These users may be registered to the computerized service or be users that interacted with the computerized service. The computerized service is executed by a computerized system and/or by a user device. The computerized system and the user device may include one or more computers, one or more processors, and the like.

User physical details of a user may include physical details, such as but not limited to, gender, age, height, weight, blood type, blood pressure, known diseases etc.

User herb consumption information of a user may include information related to his herb consumption habits. It may include herb genus consumed, plant strain used, herb quantity applied, frequency of application, consumption means used (e.g. using a herb vaporizer, a bong, a pipe etc.), consequent side effects, physical and mental condition during and after application, and the like.

User contact information of a user may include contact information for contacting the user and contact information that allows the user to contact other persons. It may include phone numbers, facsimile numbers, electronic mail addresses, user device identifiers, social network contact information, enterprise identifier of the user, and the like. Non-limiting examples of contact information formats are a Linkedin™ profile, a Facebook™ profile, Skype™ contact information, Twitter™ contact information and the like. Different persons may be associated with different formats of contact information.

Stage 610 may be followed by stage 620 of finding, in response to the users metadata, a group of users that have matching users physical details or corresponding users herb consumption information. The number of users per group of users may be two or may exceed two. A user may be a member of one or more groups of users.

Stage 620 may be followed by stage 630 of correlating between various parameters of each group of users. According to one embodiment of the invention group of users found in stage 620 represent users aged 30-35. These users may have further correlating parameters aside their age. Some of these users might be consuming the same plant strain or may be having the same side effects and so on.

Stage 630 may be followed by stage 640 of creating, in response to correlated parameters of each group of users, finding a sub-group of users that have further matching users' physical details or users herb consumption information. Each sub-group of users may have at least two matching parameters. According to another embodiment of the invention one sub-group of users may represent users aged 30-35 that consume the same Cannabis strain and experience respite of an appetite loss after application thereof.

Stage 640 may be followed by stage 650 of classifying the sub-group of users into predetermined or real-time determined categories. Each category represents essential correlated information wherein at least one matching parameter of users' physical details or users herb consumption information or any combination thereof. According to one embodiment of the invention the category represents sub-groups of users that experience a posttraumatic stress disorder (PTSD) and consume the same Cannabis strain that relieved their mental state.

Stage 650 may be followed by stage 660 of responding to the findings of the users' categories.

The responding (stage 660) may include at least one out of:
a. Providing with the top-rated strain, that will most likely adapt to him according to his physical state and his personal experience the user seeks for.
b. Providing fast and easy purchase that is adapted to the user directly through the market space 74.
c. Providing all information regrading user's top-rated strain directly from the knowledge center 73, articles, blogs, vlogs, communities etc.
d. Providing to the users of the sub-group of users an indication that they belong to the sub-group of users.
e. Providing to the users of the sub-group of users matching indications about the users' physical details and/or matching herb consumption information and/or contact information for allowing the users of the sub-group to contact each other.
f Sending an indication to a third party.
g. Providing to the users of the sub-group of users an indication about other sub-group of users that may have additional or alternative matching users' physical details and/or matching herb consumption information.

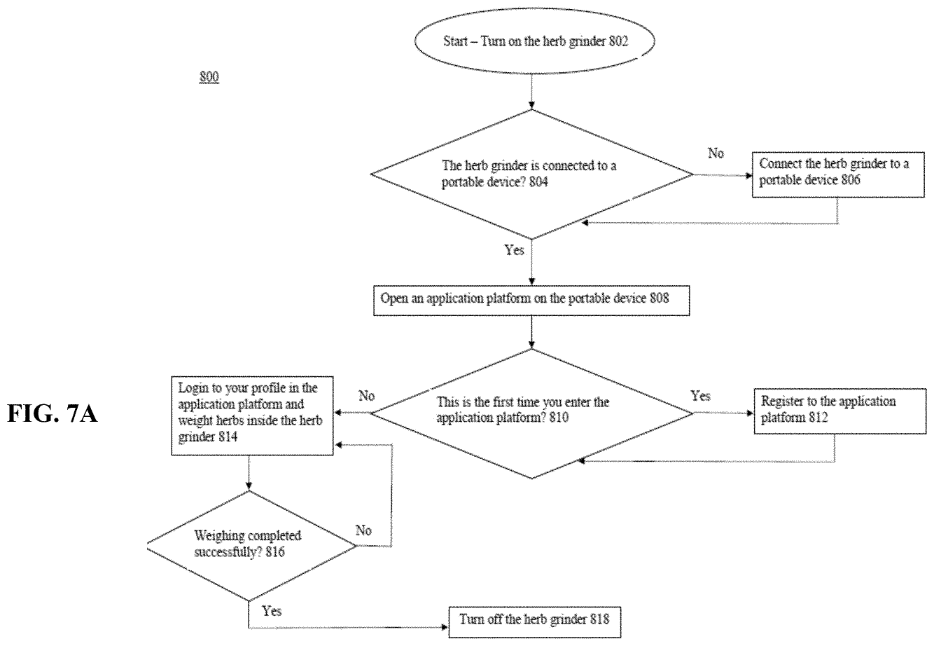

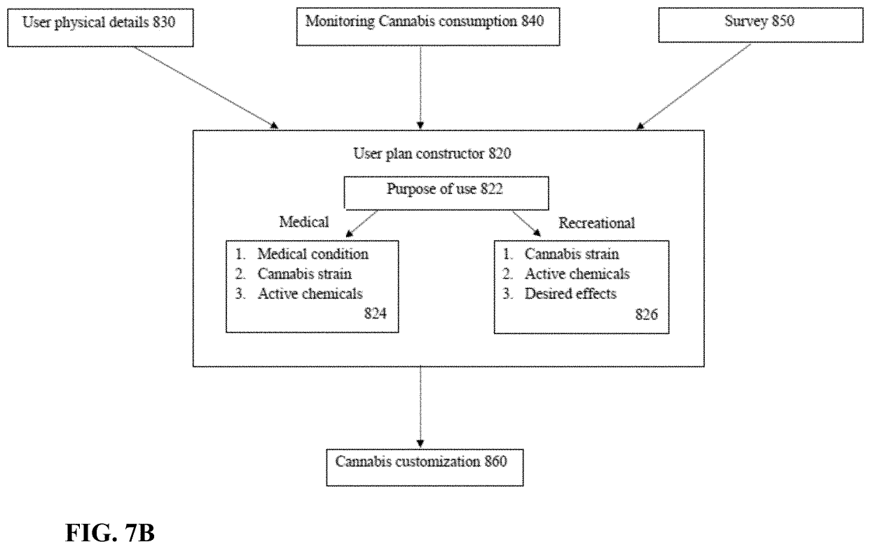

Reference is currently made to FIG. 7B, which illustrates a flowchart according to an embodiment of the invention.

The app platform (i.e. computerized system) described hereinabove is configured to store user physical details 830, monitor Cannabis consumption 840 and provide survey 850. In addition, the app platform features a user plan constructor 820 that is configured to construct a Cannabis consumption plan for the user based on his physical details, his Cannabis consumption history and his surveys. The app platform may learn users' preferences based on the various plans created by the user plan constructor 820 and provide a Cannabis customization 860 for the user.

The user physical details 830 may be as described in FIG. 5.

Monitoring Cannabis consumption 840 feature may include details the user provides the app platform such as, but not limited to, date, time, herb genus consumed, plant strain used, herb quantity applied, frequency of application, consumption means used (e.g. using a herb vaporizer, a bong, a pipe etc.), consequent side effects, physical and mental condition during and after application, and the like.

The survey 850 feature may provide the user with a consumption survey after each herb use or alternatively when applicable. The user may provide the app platform via the survey his after-use details such as positive and/or negative side effects, medical condition, comments etc. The user may give his personal feedback about his cannabis experience by rating different parameters such as: medical effects, positive effects, negative effect etc. The user may rate his experience after scaling his consumption, with a notification system, or manual rate his experience.

The plan constructor 820 may start with stage 822 wherein the user may choose his purpose of use; whether he is using Cannabis as a medical treatment or for recreational purposes.

In the event of using Cannabis as a medical treatment the plan constructor 820 may require the following details 824: users medical condition, Cannabis strain used (e.g. Indica, Sativa) and active chemicals (e.g. CBD, CBN, THC etc.) and their concentration (e.g. high, medium or low).

In the event of using Cannabis for recreational purposes the plan constructor 820 may require the following details 826: Cannabis strain used (e.g. Indica, Sativa), active chemicals (e.g. CBD, CBN, THC etc.) and their concentration (e.g. high, medium or low) and desired effects (e.g. creativity and concentration).

The user plan constructor 820 may construct Cannabis consumption plan for the user based on all his inputs as depicted hereinabove. For example, for a recreational use the user plan constructor 820 may suggest a daily plan wherein Sativa is the preferred Cannabis strain with low-CBD, low-CBN and low-THC with users experience of creativity and concentration.

Optionally, the user may choose a plan from variety of built-in programs provided by the app platform.

With the user plan constructor 820 the user may be able to adjust his Cannabis consumption accordingly.

The app platform provides Cannabis customization 860 for the user based on users physical details 830, monitoring Cannabis consumption 840 utility, survey 850 and plan constructor 820 features. The app platform may analyze corresponding metadata of all users thereof as described in FIG. 6 hereinabove. Consequently, the Cannabis customization 860 utility will suggest the user at least one alternative and/or preferred Cannabis strain that corresponds to his plan.

### Grinder Scale—Operating

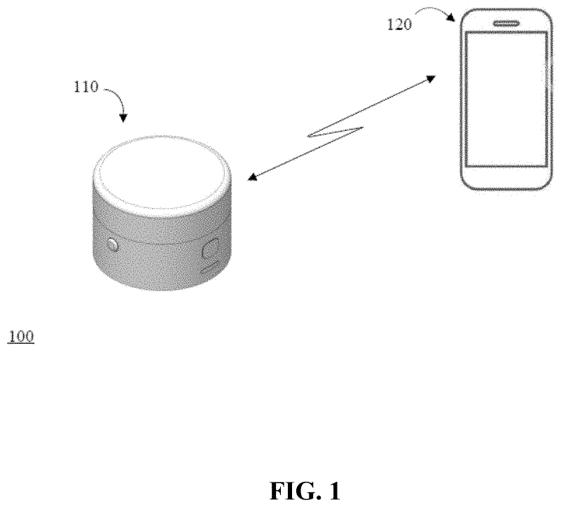

Reference is currently made to FIG. 1, which illustrates a system 100 according to an embodiment of the invention. The system 100 includes a herb grinder 110 and a portable device 120. The portable device 120 is a smart phone that incorporates a mobile application that is configured to communicate with the herb grinder.

According to other embodiments of the invention the portable device may be a personal computer (PC), a tablet PC, a set-top box (STB), a Personal Digital Assistant (PDA), a cellular telephone etc. or a desktop computer.

Reference is currently made to FIG. 7A, which illustrates a flowchart 800 according to an embodiment of the invention. In order to use the herb grinder depicted hereinabove the user should start by turning on the herb grinder 802. Next, the user should check if the herb grinder is connected to a portable device 804. If it's not connected the user should connect the herb grinder to a portable device 806 by means described hereinabove. Once the herb grinder is connected to a portable device the user should open an application platform on the portable device 808. If this is the first time the user enters the application platform 810 he should register to the application platform 812, if it's not then he should login to his personal account and thereby weight herbs inside the herb grinder 814. Once the weighing completed successfully 816 the user may turn off the herb grinder 818.

In the registration stage 812 the user must provide his phone number, gender and age. Optionally, the user may also provide his height, weight and blood type.

### Grinder Scale—Mechanical

Additionally, the invention provides a computer-implemented method for grinding herbs and monitoring consumption thereof on an app platform stored in the non-transitory storage medium of an electronic device like mobile phones or smart phones etc.

The electronic scale may include a load plate, a transducer, sensors (e.g. pressure), an electronic circuit, a microprocessor, a display (e.g. LCD screen), an internal rechargeable battery or a replaceable battery or an AC/DC adaptor, an auto-calibration software (internal and/or external), an automatic switch-off module, a power module, a communication module that may be configured to communicate with portable or stationary devices via wireless or non-wireless connection systems (e.g. Bluetooth communication, Infrared communication, radio, Wi-Fi communication, Mobile communication systems, etc.).

According to an embodiment of the invention the electronic scale may be wirelessly connected to a portable device and may have features like smartphone integration, cloud storage and weight tracking.

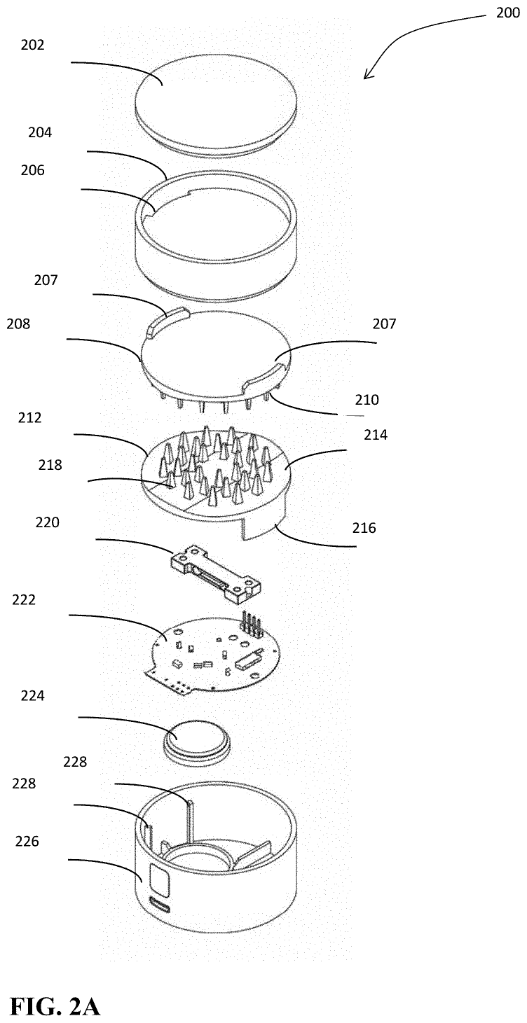

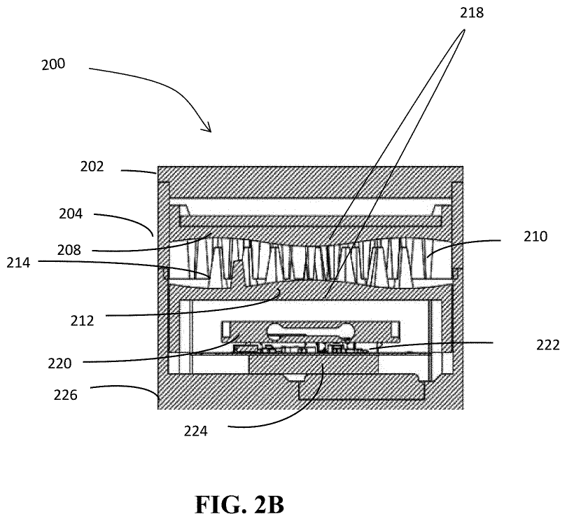

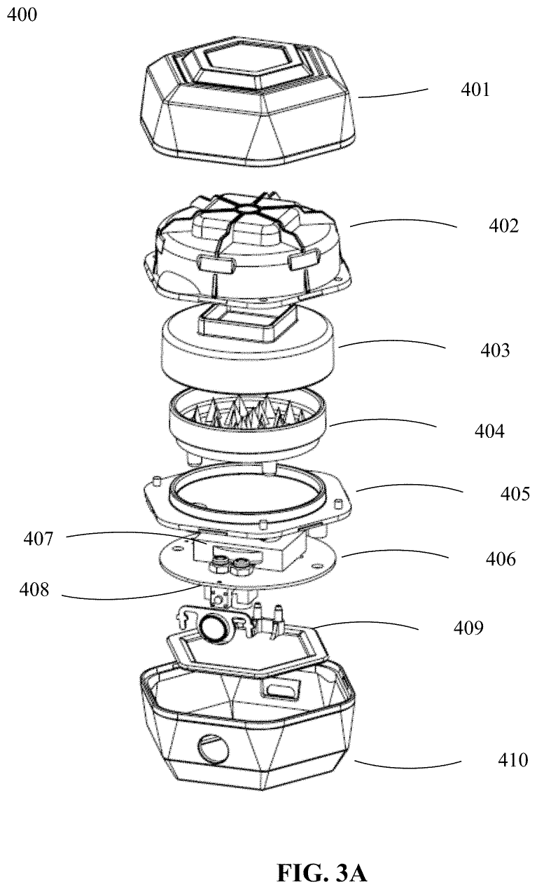

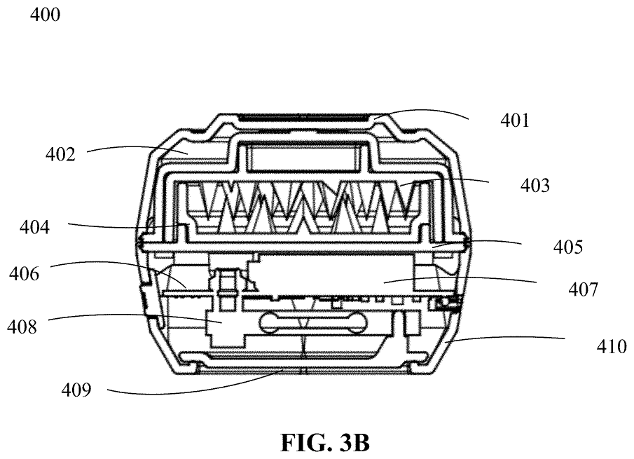

According to an embodiment of the invention the electronic scale may be integrated in the herb grinder as depicted in FIGS. 2A, 2B and 3A, 3B.

According to another embodiment of the invention the electronic scale may be an independent unit that is adapted to be coupled to any herb grinder available in the market.

According to an embodiment of the invention the electronic scale may be of 0.01 gram accuracy.

Internal structure of the herb grinder 110 is depicted in FIGS. 2A, 2B, 3A and 3B.

Reference is currently made to FIG. 2A, which is an isometric exploded view of the herb grinder 110 shown in FIG. 1, denoted 200. Herb grinder 200 includes: a lid member 202; a storage compartment 204; a top grinding plate 208; a bottom grinding plate 212; an electronic scale 220; a printed circuit board (PCB) 222; a battery 224; and a tubular casing member 226. The storage compartment 204 has an outer perimeter surface that is constructed to provide a storage area therein enclosable with the lid member 202. Said storage area eliminates the need for transporting additional storage devices. The lid member 202 is sized and shaped to fit within the storage compartment 204. The storage compartment 204 further comprising a plurality of slots configured to accommodate protruding ribs 207 which are extended outwardly of the top grinding plate 208. Such engaging structure secures the top grinding plate 208 and prevents movement thereof during grinding operation. The upper surface of the top and bottom grinding plates 208, 212 each have a plurality of upstanding spaced-apart grinding teeth 210, 214 integrally formed to the plate. Each tooth 210, 214 having a pyramidic shape. Bottom grinding plate 212 features outwardly extended protruding ribs 216 and a convex area 218 which contributes to diverse heights of the bottom grinding teeth 214 thereon. Such structure enables undulation movement of the top and bottom grinding teeth 210, 214 whereby the ground material is refined, and volume thereof is reduced; which yields a complete and consistent grind.

The tubular casing member 226 is configured to accommodate the electronic scale 220, the printed circuit board (PCB) 222 and the battery 224 therein. In addition, said tubular casing member 226 features rail members 228 provided to allow the protruding ribs 216 to be inserted and removed from the casing, and to prevent unwanted rotation thereof. The lid member 202, storage compartment 204 and the tubular casing member 226 can all be integrally assembled together so that storage compartment 204 is rotatable with respect to the tubular casing member 226 about a common axis. The upstanding spaced-apart grinding teeth 210, 214 are arranged on the upper surface of the top and bottom grinding plates 208, 212 so as to scrape against them the opposing element when the storage compartment 204 and tubular casing member 226 are engaged and are rotated relative to one another.

Reference is currently made to FIG. 2B, which is a cross section view of the herb grinder 110 shown in FIG. 1, denoted 200. Reference numerals were repeated from FIG. 2A for corresponding or analogous elements in order to provide a thorough understanding of the invention. Assembled position of the herb grinder 200 is illustrated in FIG. 2B wherein the lid member 202, the storage compartment 204 and the tubular casing member 226 are formed as a uniform body. The electronic scale 220, the printed circuit board (PCB) 222 and the battery 224 are mounted one on the top of the other respectively inside the tubular casing member 226 enclosable with the bottom grinding plate 212. The convex area 218 of the top and bottom grinding plates, 212 is clearly shown. It is to be understood that the convex area 218 is illustrated in the center of the top and bottom grinding plates 208, 212 merely for exemplary purposes while other configurations may be used. In one embodiment of the current invention the size of the top and bottom grinding teeth 210, 214 vary on the upper surface of the top and bottom grinding plates 208, 212. In other embodiments of the current invention the grinding teeth 210, 214 optionally or alternatively may have the same size and other shapes. Both grinding plates 208, 212 may be readily removed for cleaning purposes.

Reference is currently made to FIG. 3A, which is an isometric exploded view of the herb grinder 400 shown in FIG. 3B, denoted 400. Herb grinder 400 includes: a lid member 401; a housing unit 402; a top grinding plate 403; a bottom grinding plate 404; an electronic scale 408; a printed circuit board (PCB) 406; a battery; and a tubular casing member 410. The grinder is built from 2 main pieces, the upper one who is containing the upper removable teeth, 403. The bottom part which is containing the bottom removable teeth, and the electronic components, 406-408, and the scale plate, 409. The scale plate, 409, is embedded in the bottom housing to protect the scale sensor, and to operate the scale you need to set the bottom part up on the upper part, so the ledge will fit the embedded arear on the bottom part of the grinder.

Reference is currently made to FIG. 3B, which is a cross section view of the herb grinder 400 shown in FIG. 3A, denoted 400. Reference numerals were repeated from FIG. 4A for corresponding or analogous elements in order to provide a thorough understanding of the invention. Assembled position of the herb grinder 400 is illustrated in FIG. 3B wherein the lid member 401, the housing compartment 402 and the removable teeth are formed as a unitary body. The electronic scale 408, the printed circuit board (PCB) 406 and the battery 407 are mounted under the connecting plate, 405, that holds the upper teeth, 404, on the top side and holds the PCB, 406, on the bottom. 404-410 are formed as a uniform body. to operate the scale you need to set the bottom body on the top side of the upper body, in a way that the ledge on the top side will fit the bottom embedded scale plate, to calculate the pressure on the scale sensor. Both grinding plates 403, 404 may be readily removed for cleaning purposes.

### CLARIFICATIONS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention. The system may be implemented using open source techniques and in a manner to achieve maximum flexibility of the system allowing easy adaptation to many different test circumstances.

The present invention can be implemented on any communication device that has hardware components that can perform telecommunication such as (but not limited to)—computers (desktop, laptop, tablets), smart phones, personal multimedia devices, etc.

The various devices on which the applications that implement the present invention run may use one or more processors with different instruction-sets, architectures, clock-speeds, etc. and memory that may include high speed random access memory and may include non-volatile memory such as one or more magnetic disk storage devices, flash memory devices and other kinds of solid state memory devices.

The various applications that can implement the present invention run on electronic devices that may use at least one physical user interface device that provide the means of control and navigation within the operating system and applications that run on the devices include (but not limited to) touch-pads, touch screens, click wheel, keyboards, mouse and gesture recognition means.

Display means used by these devices may use LCD (liquid crystal display) technology, LED (light Emitting Diode) technology, CRT (Cathode ray tube) technology, or LPD (light emitting polymer) technology or any other display technologies.

Connectivity of these devices with networks such as the internet, an intranet and/or wireless network such as cellular telephone network, a wired or wireless local area network (LAN) and/or metropolitan area network (MAN) and/or WAN (wide area network) and other wireless communication is achieved by use of a plurality of communication standards, protocols and technologies like Global System for mobile communication (GSM), Enhanced Data GSM Environment (EDGE), wideband code division, of multiple access (W-CDMA), DLNA, LTE (Long Term Evolution), code division of multiple access (CDMA), time division multiple access (TDMA), Bluetooth, Wireless Fidelity (Wi-Fi) and/or any other suitable communication protocol, including communication protocols not yet developed as of the filing date of this document.

The present invention maybe be implemented on applications that run on a single or variety of operating system platforms including but not limited to OS X, WINDOWS, UNIX, IOS, ANDROID, SYMBIAN, LINUX, or embedded operating systems such as VxWorks.

The present invention may also be implemented to work with various web browsers including but not limited to Internet Explorer, Mozilla Firefox, Safari and Opera that access and handle various types of web pages constructed with various mark-up languages such as HTML, HTML-5, XHTML, XML, etc. and the associated CSS (cascading style sheet) files and java-script files.

The present invention implements a Graphical User Interface operable with the means for control and navigation of the user's communication device that enables the user to use the various means provided through the system.

Embodiments of the present invention may be provided as a computer program product, or software, that may include a machine-readable medium having stored thereon instructions, which may be used to program a computer system (or other electronic devices) to perform a process according to the present invention. A machine-readable medium includes any mechanism for storing or transmitting information in a form readable by a machine (e.g. a computer). For example, a machine-readable (e.g. computer readable) medium includes a machine (e.g. a computer) readable storage medium (e.g. read only memory (“ROM”), random access memory (“RAM”), magnetic disk storage media, optical storage media, flash memory devices, etc.), a machine (e.g. computer) readable transmission medium (electrical, optical, acoustical or other form of propagated signals (e.g., infrared signals, digital signals, etc.)), etc.

The computer system may further include a network interface device (NID).

The computer system may also include a video display unit (e.g. flat panel display, such as OLED, or liquid crystal display (LCD) or a cathode ray tube (CRT)), an alphanumeric input device (e.g. a keyboard), a cursor control device (e.g. a mouse), and a signal generation device (e.g. a speaker).

The computer system may further include a memory.

The memory may include a machine-accessible storage medium (or more specifically a computer-readable storage medium) on which stored one or more sets of instructions (e.g. software) embodying any one or more of the methodologies or functions described herein. The software may also reside, completely or at least partially, within the memory and/or within the processor during execution thereof by the computer system, the memory and the processor also constituting machine-readable storage media. The software may further be transmitted or received over a network via the network interface device.

While the machine-accessible storage medium is shown in an exemplary embodiment to be a single medium, the term “machine-readable storage medium should be taken to include a single medium or multiple media (e.g. centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present invention. the term “machine-readable storage medium” shall accordingly be taken to include, but not limited to, solid-state memories, and optical and magnetic media.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc. A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system. The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

The invention may also be implemented in a computer program for running on a computer system, at least including code portions for performing steps of a method according to the invention when run on a programmable apparatus, such as a computer system.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

While certain features of the invention have been illustrated and described herein, many modifications, substitutions, changes, and equivalents will now occur to those of ordinary skill in the art.

1. A system for monitoring and customizing herb consumption, comprising:
a server computer coupled to a database that is configured to store data obtained from at least one mobile computing device;
at least one digital herb scale for coupling in digital communication to a mobile computing device for monitoring herb consumption;
wherein, processing circuitry of said server computer is configured to obtain and analyze metadata obtained from at least one mobile computing device, including the actions of:
obtaining metadata of a person, wherein said metadata includes physical details and herb consumption information gathered by said at least one digital herb scale;
based on said metadata, finding a group of at least two persons that have at least one matching parameter in their physical details and herb consumption information;
in said group, finding a sub-group of at least two persons that further have an at least one additional matching parameter in their physical details and herb consumption information;
customizing a plan for herb consumption for a person based on herb consumption information of at least one other person in said sub-group.

2. The system of claim 1, wherein customizing a plan for a person is performed further based on information gathered through a survey of at least one other person in said sub-group.

3. The system of claim 1, wherein said digital herb scale is comprised of a load plate, a transducer, sensors a microprocessor, a display, a power module, and a communication module.

4. The system of claim 3, wherein said digital herb scale further comprises a lid member, a storage compartment, a top and bottom grinding plates;
wherein, said storage compartment has an outer perimeter surface that is constructed to provide a storage area therein enclosable with the lid member;
further wherein, said top and bottom grinding plates, each have a plurality of upstanding spaced-apart grinding teeth;
further wherein, said bottom grinding plate features outwardly extended protruding ribs and a convex area for undulation movement of top and bottom grinding teeth, whereby ground material is refined, and volume thereof is reduced yielding a complete and consistent grind.

5. A digital herb scale comprising a lid member having a ledge, a housing unit, a top grinding plate, a bottom grinding plate, a scale plate having an embedding that fits said ledge of said lid member, an electronic scale arranged such that to measure pressure on said scale plate, a microprocessor, a power module, a communication module; wherein, weight of herbs on said digital herb scale is derived from pressure on said scale plate from said lid member that said scale plate is placed thereupon.

6. A method for monitoring and customizing herb consumption, comprising the steps of:
obtaining metadata of a person, wherein said metadata includes physical details and herb consumption information gathered by a digital herb scale electronically coupled to at least one mobile computerized device;
based on said metadata, finding a group of at least two persons that have at least one matching parameter in their physical details and herb consumption information;
in said group finding a sub-group of at least two persons that have further an additional at least one matching parameter in their physical details and herb consumption information;
customizing a plan for herb consumption for a person based on herb consumption information of at least one other person in said sub-group.

7. The method of claim 6, wherein customizing a plan for a person is performed based on further information gathered through a survey of at least one other person in said sub-group.
